(19) United States
(12) Patent Application Publication
(10) Pub. No.: US 2026/0345513 A1
(43) Pub. Date: Nov. 5, 2026

(54) STABLE TRANSDERMAL DRUG DELIVERY KIT, PREPARATION METHOD THEREFOR, AND USE THEREOF

(71) Applicant: NOVASTAGE PHARMACEUTICALS (SHENZHEN), LTD., Shenzhen City, Guangdong (CN)

(72) Inventor: Jiansheng TANG, Shenzhen, Guangdong (CN)

(21) Appl. No.: 19/102,645

(22) PCT Filed: Jan. 4, 2023
(86) PCT No.: PCT/CN2023/070545

§ 371 Date: Feb. 10, 2025

(51) Int. Cl. A61K 9/70; A61K 45/06

(52) U.S. Cl. CPC A61K 9/7061; A61K 45/06

(57) ABSTRACT

A stable transdermal drug delivery kit, a preparation method therefor, and a use thereof. The transdermal kit comprises a transdermal drug delivery patch, and the transdermal drug delivery patch comprises: 1) a backing layer; 2) a matrix layer containing a drug or a pharmaceutically acceptable salt thereof dispersed in a non-crystalline or partially crystalline state in the matrix layer, an antioxidant, and a pressure-sensitive adhesive, and optionally containing a stabilizer for inhibiting drug crystallization, wherein the drug can be oxidized and degraded, and is preferably oxidized and degraded to generate nitrogen oxide impurities; and 3) a release layer, wherein the transdermal drug delivery patch is packaged in a packaging container, and the packaging container is filled with inert gas.

### TECHNICAL FIELD

The present invention relates to a stable transdermal drug delivery kit. More specifically, the present invention relates to a transdermal drug delivery kit for a drug or a pharmaceutically acceptable salt thereof, and a preparation method therefor and use thereof.

### BACKGROUND ART

The transdermal drug delivery route is superior to the oral drug delivery route, and maintains drug concentration in the blood at a constant level by continuously delivering drugs to the systemic blood system. The transdermal drug delivery route not only reduces the peak-to-trough fluctuations in drug concentration in the blood, but also avoids the first-pass effect. In addition, since the transdermal drug delivery route avoids direct contact of drugs and excipients with the gastrointestinal system, side effects such as nausea and vomiting that are often associated with the oral drug delivery route are significantly reduced or eliminated. Another advantage of the transdermal drug delivery route lies in being not affected by diet. Drug delivery can be easily terminated when necessary by removing the transdermal patch from the skin. Furthermore, transdermal patches improve patient compliance by reducing the frequency of drug delivery. This is especially important for elderly patients and pediatric patients.

In recent years, the advantages of transdermal drug delivery have enabled many drugs to be effectively delivered via a transdermal route. These advances include the development of many physical methods for increasing skin permeability and facilitating transdermal drug delivery, e.g., using iontophoresis, electroporation, ultrasound, or microneedle. However, drugs that can be effectively and safely applied through the skin for 7 days or more without causing skin adhesion, skin irritation or sensitization are still limited.

To date, different transdermal therapeutic systems (TTS) have been described. For example, WO 99/49852, WO 02/15903, WO 94/07468 and WO 99/49852 disclose rotigotine transdermal drug delivery systems; U.S. Pat. No. 5,891,461 and 2007/0148218A1 disclose olanzapine transdermal drug delivery systems.

Some matrix materials of transdermal drug delivery formulations contain oxides, which can oxidize and degrade unstable drugs, thereby affecting the long-term stability of transdermal patches. The inventors have found that a peroxide excipient such as an acrylic pressure-sensitive adhesive, povidone, copovidone and crospovidone CL-M contained in a matrix layer of a transdermal patch can accelerate the oxidative degradation of a drug, for example, to generate nitrogen oxides.

In general, stabilizing an amorphous state of a medicinal substance in a pharmaceutical dosage form (including a transdermal system) is very difficult. The amorphous form of a drug is only relatively stable and is easily converted into a crystalline form in a transdermal formulation. The transdermal formulations of olanzapine and rotigotine both have the defect of changing the crystalline state of the drug. If drug crystals form in the matrix during long-term storage, the growth of the crystals may lead to a reduced release rate of the drug, with the risk of eventually falling below a specified value. The use of substances for inhibiting drug crystallization, such as povidone, copovidone, crospovidone and other matrix materials containing oxides or peroxides, may lead to problems of drugs being oxidized and degraded.

Therefore, there is an urgent need for a transdermal drug delivery system that can keep drugs stable for a long period of time and meet the requirements of ICH Q3B.

### SUMMARY OF THE INVENTION

It has been found in the present application that some matrix materials of transdermal drug delivery formulations contain oxides or peroxides, which can oxidize and degrade unstable drugs, thereby affecting the long-term stability of transdermal patches. For example, it has been found in the present application that a peroxide excipient such as an acrylic pressure-sensitive adhesive, and a stabilizer for inhibiting crystallization such as povidone, copovidone and crospovidone CL-M contained in a matrix layer of a transdermal patch can both accelerate the oxidative degradation of a drug. The oxidative degradation products include toxic nitrogen oxides. For example, olanzapine, rotigotine and risperidone in a transdermal patch are all easily affected by oxides or peroxides in a matrix material, thereby generating oxidative degradation impurities such as nitrogen oxides.

Under conventional storage conditions for pharmaceutical products, rotigotine in a transdermal patch formulation is easily degraded by peroxides to produce N-oxides (EP impurity E), which are further decomposed into other related substances (such as EP impurity C, EP impurity B, and EP impurity K), rendering the transdermal patch formulation unusable.

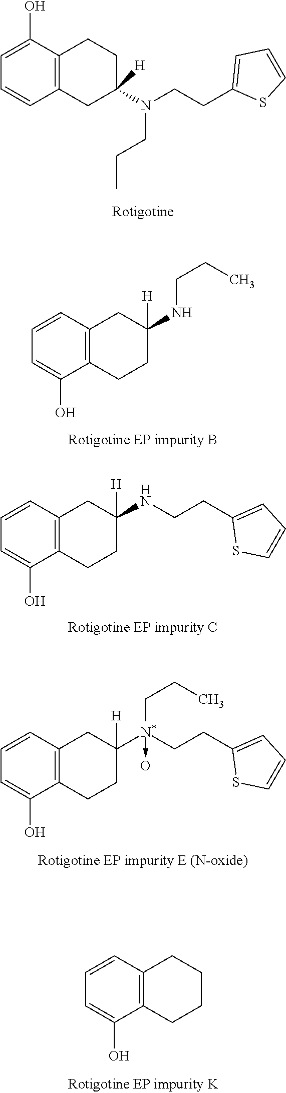

Under conventional storage conditions for pharmaceutical products, the oxidative degradation products of olanzapine in a transdermal patch formulation include olanzapine EP impurity B and olanzapine EP impurity D (N-oxide).

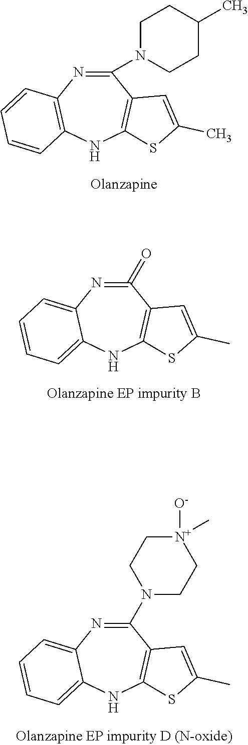

Under conventional storage conditions for pharmaceutical products, the oxidative degradation products of risperidone in a transdermal patch formulation include risperidone N-oxide.

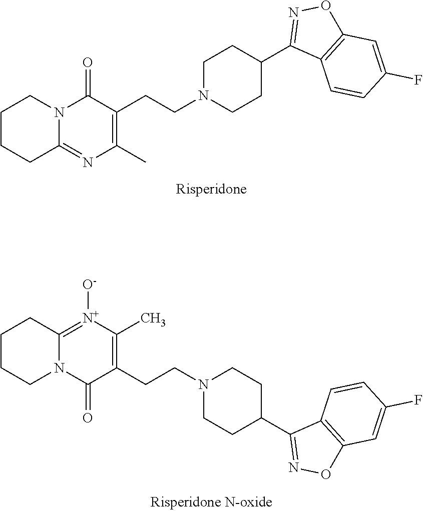 [SMILES: Cc1nc2n(c(=O)c1CCN1CCC(c3noc4cc(F)ccc34)CC1)CCCC2 ; Cc1c(CCN2CCC(c3noc4cc(F)ccc34)CC2)c(=O)n2c([n+]1[O-])CCCC2]

The present inventors have also found that adding an antioxidant combination to a drug matrix layer of a transdermal patch can inhibit the degradation of the drug by oxides and reduce the generation of related substances.

The present inventors have also unexpectedly found that filling a packaging container (e.g., a packaging bag) of a patch formulation, to which an antioxidant is added as a matrix, with an inert gas such as nitrogen can further inhibit the oxidative degradation of the drug.

The present applicants have surprisingly found that, on the basis of filling with nitrogen, adding a hygroscopic agent not in contact with the matrix layer of the patch to a packaging can further inhibit the oxidative degradation of the drug.

The present inventors have also unexpectedly found that on the basis of filling with nitrogen or a combination of filling with a gas and a hygroscopic agent in contact with the matrix layer of the patch, further addition of an oxygen scavenger not in contact with the matrix layer of the patch to the packaging bag does not result in decreased stability.

An object of the present invention is to provide a stable matrix-type transdermal drug delivery formulation and a kit thereof, which, after storage for a period of time, maintain the content of related substances at an extremely low level.

An object of the present invention is to provide a stable matrix-type transdermal drug delivery formulation and a kit thereof, which can inhibit the drug from being degraded by oxides or peroxides in the matrix.

Another object of the present invention is to provide a matrix-type transdermal drug delivery formulation and a kit thereof, which, after storage for a period of time, have no changes in the crystalline state of the drug. Preferably, the drug in the transdermal patch does not crystallize, and the proportion of crystals does not change significantly.

Another object of the present invention is to provide a matrix-type transdermal drug delivery formulation and a kit thereof, which, after storage for a period of time, can continuously deliver the drug or pharmaceutically acceptable salt thereof at a therapeutically effective blood drug level over a prolonged period of time.

Another object of the present invention is to provide a method for preparing a matrix-type transdermal drug delivery formulation and a kit thereof. After storage for a period of time, the matrix-type transdermal drug delivery formulation and kit thereof prepared by the method maintain the content of related substances at an extremely low level. After storage for a period of time, the crystallization state of the drug in the transdermal patch does not change. Preferably, the drug in the transdermal patch does not crystallize, or the proportion of crystals does not change significantly. After storage for a period of time, the transdermal patch can continuously deliver the drug or pharmaceutically acceptable salt thereof at a therapeutically effective blood drug level over a prolonged period of time.

Another object of the present invention is to provide a method for treating or preventing a disease, comprising administering a therapeutically effective amount of a transdermal drug delivery kit to a subject in need thereof.

Another object of the present invention is to provide the use of a therapeutically effective amount of a matrix-type crystallization-inhibiting transdermal drug delivery kit in the preparation of a medicament for treating or preventing a disease.

### DETAILED DESCRIPTION OF THE INVENTION

A first aspect of the present invention provides a transdermal drug delivery kit, comprising a transdermal drug delivery patch, wherein the transdermal drug delivery patch comprises:
1) a backing layer;
2) a matrix layer containing a drug or a pharmaceutically acceptable salt thereof dispersed in a non-crystalline or crystalline state in the matrix layer, an antioxidant, and a pressure-sensitive adhesive, and optionally containing a stabilizer for inhibiting drug crystallization, wherein the drug can be oxidized and degraded, and is preferably oxidized and degraded to generate nitrogen oxide impurities; and
3) a release layer;
wherein the transdermal drug delivery patch is packaged in a packaging container.

In some embodiments, the packaging container is filled with an inert gas. The inert gas is preferably nitrogen. Preferably, nitrogen is filled to reduce oxygen to below about 1.0%, and more preferably, nitrogen is filled to reduce oxygen to below about 0.5%, 0.2%, 0.15%, 0.1%, 0.05% and 0.01%.

In some embodiments, a hygroscopic agent not in contact with the matrix layer of the patch is further added to the packaging container. The hygroscopic agent is selected from one or more of calcium chloride, calcium oxide, barium oxide, calcium sulfate, molecular sieve, sodium sulfate, magnesium sulfate, silicon dioxide, silica gel, potassium hydroxide, potassium carbonate and phosphorus pentoxide.

In some embodiments, an oxygen scavenger not in contact with the matrix layer of the patch is further added to the packaging container. The oxygen scavenger comprises a main component selected from a combination of iron powder and sodium chloride, a combination of ferrous carbonate and metal halide, and a combination of ascorbate and sodium bicarbonate, preferably a combination of iron powder and sodium chloride, e.g., iron-based oxygen scavenger used in the examples.

In some embodiments, the hygroscopic agent and/or oxygen scavenger are separately packaged in a breathable film. Preferably, the hygroscopic agent and/or oxygen scavenger packaged in the breathable film are separately adhered to or placed on a heat-sealing layer in the packaging container, or are uniformly contained in the heat-sealing layer.

In some embodiments, the packaging container has low oxygen permeability and low moisture permeability, and is preferably a metal tray, a plastic tray, or a packaging bag made of a multi-layer laminated material comprising a plastic or paper layer, an aluminum foil layer and a heat-sealable inner layer.

In some embodiments, the stabilizer for inhibiting drug crystallization and/or the pressure-sensitive adhesive contains a peroxide. Preferably, the peroxide-containing stabilizer for inhibiting drug crystallization is selected from insoluble crospovidone, an acrylic acid polymer or a methacrylic acid polymer. The peroxide-containing pressure-sensitive adhesive is an acrylic pressure-sensitive adhesive. further preferably, the insoluble crospovidone is selected from one or more of insoluble crospovidone CL-M, crospovidone CL, crospovidone CL-F and crospovidone CL-SF. Further preferably, the peroxide-containing acrylic acid polymer or methacrylic acid polymer for inhibiting drug crystallization is selected from one or more of Eudragit E100, Eudragit PO, Plastoid B, Eudragit S, Eudragit L and Eudragit L-55.

In some embodiments, on the basis of the peroxide-containing stabilizer for inhibiting drug crystallization, the stabilizer for inhibiting drug crystallization further comprises a second stabilizer for inhibiting drug crystallization which is different from a first stabilizer for inhibiting drug crystallization, wherein the second stabilizer for inhibiting drug crystallization is selected from one or more of povidone, copovidone, crospovidone, carbohydrate polymers such as hydroxymethyl cellulose, methyl hydroxymethyl acetate, hydroxymethylcellulose phthalate, 3-hydroxypropyl cyclodextrin, ethyl cellulose, methyl cellulose, Katasong, hyaluronic acid, pectin, carboxymethyl cellulose, alginic acid and carrageenan; acrylic acid or methacrylic acid polymers such as Eudragit E100, Eudragit PO, Plastoid B, Eudragit S, Eudragit L and Eudragit L-55; more preferably, the second stabilizer for inhibiting drug crystallization is selected from one or more of soluble povidone, Eudragit E100 or Eudragit PO; the povidone is preferably selected from one or more of povidone K30, povidone K90, povidone K12, povidone K17, povidone K25, plasdone K29/32 and copovidone VA64.

In some embodiments, the antioxidant is selected from one or more of ascorbic acid, ascorbyl palmitate, butylated hydroxyanisole, butylated hydroxytoluene, hypophosphorous acid, monothioglycerol, potassium metabisulfite, propyl gallate, sodium bisulfite, sodium formaldehyde sulfoxylate, sodium metabisulfite, sodium thiosulfite, sodium thiosulfite and DL-α-tocopherol. Preferably, the antioxidant further comprises a chelating agent as a synergistic antioxidant, including one or more of citric acid, tartaric acid, calcium disodium edetate, disodium edetate and EDTA.

In some embodiments, a combination of antioxidants in the matrix is selected from: a combination of ascorbyl palmitate, sodium metasulfite and DL-α-tocopherol (vitamin E), or a combination of ascorbyl palmitate, sodium metasulfite, DL-α-tocopherol (vitamin E) and BHT.

In some embodiments, the weight ratio of the drug to insoluble povidone is no more than 9:40, preferably 9:1-9:24, 9:5-9:22.8, 9:5, 9:6.8, 9:8, 9:10, 9:12, 16.5, 9:18, 9:20, 9:21, 9:22.8, 9:24 and 9:27.

In some embodiments, the weight ratio of the drug to soluble povidone is 9:0.5-9:10, and specifically, can be selected from 9:1, 9:2, 9:3, 9:4, 9:5, 9:6, 9:7, 9:8, 9:9 and 9:10.

In some embodiments, the matrix layer comprises the following components, relative to the total weight of the matrix layer:
1) the drug or pharmaceutically acceptable salt thereof at an amount of 3%-30%;
2) the stabilizer for inhibiting drug crystallization at an amount of 0%-40%;
3) the antioxidant at an amount of 0.001%-10%;
4) the pressure-sensitive adhesive at an amount of 30%-95%;
5) a tackifier at an amount of 0%-50%;
6) a skin penetration enhancer at an amount of 0%-30%;
7) a cohesion-promoting additive at an amount of 0%-20%;
the total amount of each component in the matrix layer is 100%.

In some embodiments, the pressure-sensitive adhesive is selected from one or more of an acrylic adhesive, a silicone adhesive, an acrylic-silicone copolymer adhesive, a polybutylene adhesive, a styrene-isoprene-butylene copolymer and a styrene-butadiene-styrene copolymer.

In some embodiments, the tackifier is selected from silicone oil, light mineral oil, polybutylene, triethyl citrate, terpene and a mixture thereof.

In some embodiments, the drug that can be degraded to generate nitrogen oxide impurities is selected from olanzapine, rotigotine, donepezil, almotriptan, aripiprazole, apixaban, asenapine, baricitinib, bisoprolol, blonanserin, buprenorphine, cariprazine, dextroamphetamine, dexmedetomidine, eletriptan, escitalopram, fingolimod, frovatriptan, granisetron, lasmiditan, lenalidomide, naratriptan, oxybutynin, ozanimod, piroxicam, pramipexole, risperidone, rizatriptan, ropinirole, scopolamine, sumatriptan, tulobuterol and zolmitriptan, or a pharmaceutically acceptable salt thereof.

In some embodiments, the content of the drug or pharmaceutically acceptable salt thereof is 5% to 20%, preferably 5% to 15%, or 5% to 12% of the total weight of the matrix layer.

In some embodiments, the content of the stabilizer for inhibiting drug crystallization is 0% to 40%, preferably 6% to 40%, 6% to 30%, 6% to 20%, 6.7%-20%, 6.70%, 8.2%, 9.2%, 10%, 12.5%, 13%, 16.65%, 17.50%, 19.00%, 20.00%, 25% and 30% of the total weight of the matrix layer.

In some embodiments, the amount of crospovidone for inhibiting drug crystallization is 6% to 40%, preferably 6% to 30%, 6% to 20%, 6.7%-20%, 6.70%, 8.2%, 9.2%, 10%, 12.5%, 13%, 16.65%, 17.50%, 19.00%, 20.00%, 25% and 30%.

In some embodiments, the amount of povidone for inhibiting drug crystallization is 0%-10%, and can be specifically selected from 0%, 1%, 2%, 3%, 4%, 5%, 6%, 7%, 8%, 9% and 10%.

In some embodiments, the content of the pressure-sensitive adhesive is 35% to 90%, preferably 40% to 90%, 70% to 90%, 65%, 70%, 75%, 80% and 90% of the total weight of the matrix layer.

In some embodiments, the amount of the tackifier is 0%-50%, preferably 0%-30% and 0%-28% of the total weight of the matrix layer.

In some embodiments, the amount of each antioxidant is 0.001%-10%, preferably 0.0015%-5%. The specific amount of each antioxidant can be selected from 0.0015%, 0.002%, 0.004%, 0.006%, 0.008%, 0.01%, 0.02%, 0.03%, 0.04%, 0.05%, 0.06%, 0.07%, 0.08%, 0.09% and 0.1%.

In some embodiments, the total amount of multiple antioxidants is 0.001%-10%, and can be specifically selected from 5%, 1%, 0.5%, 0.25%, 0.1%, 0.075%, 0.05%, 0.025%, 0.01%, 0.0075%, 0.005%, 0.0025%, 0.002% and 0.0015%.

In some embodiments, the weight ratio of the drug to the tackifier is 9:25-9:40, preferably 9:30-9:35.

Another aspect of the present invention provides a method for preparing the aforementioned transdermal drug delivery kit, comprising the following steps:
step 1: dissolving an antioxidant in a solvent, dissolving or suspending a stabilizer for inhibiting drug crystallization in the solvent, and mixing for 0.1 hours to 24 hours; preferably, the solvent includes but is not limited to one or more of toluene, ethanol, isopropanol, dimethylacetamide, dimethyl sulfoxide and n-heptane, and more preferably, the solvent is toluene, ethanol, isopropanol, or a mixed solvent thereof; Further, the following steps are comprised: step 1: adding components other than a drug;
step 2: adding the drug or a pharmaceutically acceptable salt thereof, and mixing and dissolving until the drug or pharmaceutically acceptable salt thereof is dispersed in a non-crystalline state or in a crystalline state;
step 3: adding a pressure-sensitive adhesive and mixing uniformly to obtain a drug wet mixture;
step 4: coating the drug wet mixture onto a release film;
step 5: drying to remove the solvent so as to obtain a release film/matrix layer laminated material;
step 6: laminating the matrix layer onto the backing layer, and performing die-cutting on a composite film to obtain individual patches;
step 7: filling a packaging container with an inert gas, and sealing and packaging the inert gas, a hygroscopic agent and/or an oxygen scavenger and the cut individual transdermal patch into the container.

In some embodiments, the solvent in step 1 includes but is not limited to one or more of toluene, ethanol, isopropanol, dimethylacetamide, dimethyl sulfoxide and n-heptane, preferably toluene, ethanol, isopropanol, n-heptane, or a mixed solvent thereof.

Unexpectedly, it has been found in the present application that during the preparation of a transdermal patch, heating a drug-adhesive wet mixture to above room temperature prior to coating can obtain a patch that does not contain drug crystals in a matrix layer. In contrast, if the wet mixture is not heated, crystals form in the matrix layer of the patch.

In some embodiments, the step 2 comprises the step of heating to 35 °C-65 °C for achieving a dissolved state. The heating in the step 2 is preferably performed at 45 °C-55 °C, 45 °C, 46 °C, 47 °C, 48 °C, 49 °C, 50 °C, 51 °C, 52 °C, 53 °C, 54 °C and 55 °C.

In some embodiments, the step 3 involves adding a pressure-sensitive adhesive that has been heated to 35 °C-65 °C and mixing uniformly to obtain a drug wet mixture. The heating in the step 3 is preferably performed at 45 °C-55 °C, 45 °C, 46 °C, 47 °C, 48 °C, 49 °C, 50 °C, 51 °C, 52 °C, 53 °C, 54 °C and 55 °C.

In some embodiments, the step 4 involves coating the drug wet mixture that has been heated to 35 °C-65 °C onto a release film. The heating in the step 4 is preferably performed at 45 °C-55 °C, 45 °C, 46 °C, 47 °C, 48 °C, 49 °C, 50 °C, 51 °C, 52 °C, 53 °C, 54 °C and 55 °C.

In some embodiments, the drug or pharmaceutically acceptable salt thereof, the pressure-sensitive adhesive, the skin penetration enhancer and the antioxidant are pre-dried.

In some embodiments, the hygroscopic agent and/or oxygen scavenger are separately packaged in a breathable film. Preferably, the hygroscopic agent and/or oxygen scavenger packaged in the breathable film are separately adhered to or placed on a heat-sealing layer in the packaging container, or are uniformly contained in the heat-sealing layer.

Another aspect of the present invention provides the use of the aforementioned transdermal drug delivery kit in the preparation of a medicament for treating or preventing a disease.

Another aspect of the present invention provides a method for treating or preventing a disease, comprising administering a therapeutically effective amount of the aforementioned transdermal drug delivery kit to a subject in need thereof.

In a further embodiment, the transdermal drug delivery kit is administered once every 1 day, every 3 days, every 7 days, every 14 days, every 21 days, or every 28 days.

In a further embodiment, the transdermal drug delivery kit delivers about 1 mg to about 18 mg of the drug or pharmaceutically acceptable salt thereof to the blood circulation system of the subject per day, preferably delivers about 1 mg to about 12 mg of the drug or pharmaceutically acceptable salt thereof to the blood circulation system of the subject per day.

In a further embodiment, the transdermal drug delivery kit continuously delivers olanzapine or a pharmaceutically acceptable salt thereof at a therapeutically effective blood drug level over 24 hours to 28 days.

### DEFINITIONS

As used herein, the term “pharmaceutically acceptable salt” refers to those salts that are suitable for contact with subjects (e.g., human subjects) within reasonable medical judgment without excessive toxicity, irritation, allergic reaction, etc., have a reasonable benefit/risk ratio, and are effective for their intended use.

The “pharmaceutically acceptable salt” mentioned in the present invention includes inorganic acid addition salts and organic acid addition salts, can be prepared in situ during the final separation and purification of compounds, or can be prepared by reacting a purified compound in the form of a free base (for example, rotigotine) with an appropriate organic acid or inorganic acid separately and separating the salts thus formed.

As used herein, the term “therapeutically effective amount” refers to the amount of a compound or molecule of the present invention that, when administered to a subject, (i) treats or prevents a specific disease, disorder or condition, (ii) attenuates, improves or eliminates one or more symptoms of the specific disease, disorder or condition, or (iii) prevents or delays the onset of one or more symptoms of the specific disease, disorder or condition described herein.

As used herein, the term “about” means plus or minus 10% of the indicated number. For example, “about 10%” may represent a range of 9% to 11%, and “about 1” may represent 0.9-1.1.

As used herein, the term “treatment” refers to clinical intervention that attempts to alter the natural course of the individual being treated, and may be performed for prevention or in the course of clinical pathology. Desirable effects of treatment include, but are not limited to, preventing occurrence or recurrence of diseases, alleviating symptoms, weakening any direct or indirect pathological consequences of diseases, preventing metastasis, decreasing the rate of disease progression, ameliorating or alleviating the disease state, and reliving or improving prognosis.

As used herein, the term “insoluble crospovidone” is a high-molecular weight water-insoluble polymer obtained by subjecting N-vinyl-2-pyrrolidone to a cross-linking reaction, is a white or nearly white powder, is odorless, has good fluidity, is insoluble in water and various solvents, and is also insoluble in strong acids or strong bases. Non-limiting examples of “insoluble crospovidone” include CL-M, crospovidone CL, crospovidone CL-F and crospovidone CL-SF.

As used herein, “iron-based oxygen scavenger” is an iron-based deoxidizer, which comprises a combination of iron powder and sodium chloride as a main component.

The pressure-sensitive adhesive includes, but is not limited to, an acrylic adhesive, a silicone adhesive, an acrylic-silicone copolymer adhesive, a polybutylene adhesive, a styrene-isoprene-butylene copolymer and a styrene-butadiene-styrene copolymer, or a combination of two or more of the adhesives. Examples of acrylic adhesives are Duro-Tak® 387-2516, Duro-Tak®k 387-4280, Duro-Tak® 387-2287, Duro-Tak® 387-2510, Duro-Tak® 87-2196, Duro-Tak® 387-2051, Duro-Tak® 387-2052, Duro-Tak® 387-2054, Duro-Tak® 87-2194, Duro-Tak® 87-235A, Duro-Tak® 87-900A, Duro-Tak® 87-9301, Duro-Tak® 87-4098, GEFVA GMS® 788, GEFVA GMS® 9073, Duro-Tak® 387-2353, Duro-Tak® 87-2074, Duro-Tak® 87-2852, Duro-Tak® 87-2054, GELVA®-73 Tak® 80-1196, Duro-Tak® 87-2070, Duro-Tak® 87-2979, Duro-Tak® 87-2888 and Duro-Tak® 87-2296 from Henkel Corporation (Bridgewater, New Jersey); silicone adhesives BIO-PSA® 7-4401, BIO-PSA® 7-4402, BIO-PSA® 7-4501, BIO-PSA® 7-4502, BIO-PSA® 7-4601, BIO-PSA® 7-4602, SRS7-4502, SRS7-4501, SRS7-4502, SRS7-4602, BIO-PSA® 7-4101, BIO-PSA® 7-4102, BIO-PSA® 7-4103, BIO-PSA® 7-4201, BIO-PSA® 7-4202, BIO-PSA® 7-4203, BIO-PSA® 7-4301, BIO-PSA® 7-4302, BIO-PSA® 7-4302, BIO-PSA® 7-6101, BIO-PSA® 7-6102, BIO-PSA® 7-6301 and BIO-PSA® 7-6302 from Dupont (Midland, MI). Examples of polybutylene adhesives are combinations of two or more of low-molecular weight polyisobutylenes (Oppanol B10, Oppanol B11, and Oppanol B12 from BASF), medium-molecular weight polyisobutylenes (Oppnaol B50, Oppanol B80, and Oppnaol N80), and high-molecular weight polyisobutylenes (Oppanol B100, Oppanol B150, Oppanol N100, and Oppanol N150). An example of a styrene-isoprene-butylene copolymer is D1161 (Kraton).

As used herein, the term “backing layer” serves as the upper surface of a transdermal patch and provides flexibility to the patch as a main structural element. Preferably, the backing layer is substantially impermeable to a transdermally administered pharmaceutical composition. The backing layer is preferably made from a sheet or film of flexible elastic material. The backing layer is preferably air-impermeable. The backing layer used in the patch of the present invention is preferably made of a flexible, biocompatible material that mimics the elastic properties of the skin and conforms to the skin during movement. Non-occlusive backing layers allow the area to breathe (i.e. promote water vapor transmission across the skin surface), whereas occlusive backing layers reduce air/vapor penetration. Preferably, the backing layer of the matrix-type transdermal drug delivery kit is occlusive. Preferably, the backing layer contains synthetic polymers such as polyolefins, polyesters, polyethylene, polyvinylidene chloride and polyurethanes. Preferably, the thickness of the backing layer is from about 0.5 mil to about 5 mil; more preferably, the thickness of the backing layer is from about 1 mil to about 3 mils. Preferably, the oxygen transmission rate is from about 2 cc/m/24 hr to about 100 cc/m/24 hr, and more preferably, the oxygen transmission rate is from about 70 g/m/24 hr to about 90 g/m/24 hr. Preferably, the MVTR is from about 0.1 g/m/24 hr to about 50 g/m/24 hr, more preferably, the MVTR is from about 0.3 g/m/24 hr to about 30 g/m/24 hr. In a preferred embodiment, the backing layer is an occlusive polyester film layer of approximately 2.0 mil thick (commercially available as Scotchpak 9733, Scotchpak 9735 and Scotchpak 9723, 3M Drug Delivery Systems, St. Paul Minn.). Scotchpak 9733 is composed of polyester and medium density polyethylene/ethylene vinyl acetate heat-sealing layer, and the laminate is translucent, conformable, occlusive and heat-sealable. More preferably, the backing layer comprises a laminated material containing an aluminum foil layer between polymer film layers, such as Scotchpak 9738 and Scotchpak 1109. When the patch is applied to the skin, the aluminum layer prevents light from coming into contact with the photosensitive rotigotine.

As used herein, the term “release layer” includes, but is not limited to, silicone-coated polyester release liner available from many suppliers, fluoropolymer-coated polyester release liner from 3M, and fluorosilicone-coated polyester release liner.

The present invention has the advantages and technical effects as follows.

1. The present inventors have reduced the generation of degradation impurities by adding a combination of antioxidants to a drug-matrix layer.

2. The present inventors have further reduced the generation of degradation impurities by removing air from a packaging container (e.g., a pouch) and filling with an inert gas.

3. The present inventors have reduced the impurities formed in an oxidation pathway in the drug patch by packaging an antioxidant and a hygroscopic agent that are not in contact with the matrix layer of the patch, or a combination of the two. Unexpectedly, the hygroscopic agent reduces the generation of oxidative impurities. On the basis of removing air and filling with an inert gas and the hygroscopic agent, further addition of an oxygen scavenger not in contact with the patch does not result in decreased stability.

4. The inventors have found that during the preparation of a transdermal patch, heating a drug-adhesive wet mixture to above room temperature prior to coating can obtain a patch that does not contain drug crystals in a matrix layer.

5. The present inventors have reduced impurities by pre-drying bulk drugs and excipients prior to mixing.

Therefore, the present invention obtains a stable transdermal drug delivery kit, which can be stored for a long period of time under conventional conditions, has no crystallization of the drug dispersed in the matrix of the patch, and maintains the content of related substances at an extremely low level.

### DETAILED DESCRIPTION OF EMBODIMENTS

In order to make the objectives, technical solutions and advantages of the embodiments of the present invention clearer, the technical solutions in the embodiments of the present invention will be described clearly and completely below. Apparently, the described embodiments are module embodiments of the present invention, but not all of the embodiments. Elements and features described in one embodiment of the present invention may be combined with elements and features illustrated in one or more other embodiments. It should be noted that for purposes of clarity, representation and description of components and processes known to those of ordinary skill in the art that are not relevant to the present invention have been omitted from the description. Based on the embodiments of the present invention, all the other embodiments obtained by those of ordinary skill in the art without any inventive efforts should fall within the scope of protection of the present invention.

### ABBREVIATIONS

PVP K90=povidone K90, crospovidone=crospovidone CL-M, ascorbyl palmitate, Sodium Metasulfite is sodium metasulfite, BHT is 2,6-di-tert-butyl-p-cresol, Bio-PSA 7-4202 is a pressure-sensitive adhesive, Bio-PSA 7-4302 is a pressure-sensitive adhesive, Bio-PSA SRS7-4502 is a pressure-sensitive adhesive, Oppanol B12 SFN is a polyisobutylene adhesive, Oppanol N100 is a polyisobutylene adhesive, Eudragit E100 is an acrylic polymer stabilizer for inhibiting drug crystallization.

### Comparative Example A1

Each component was weighed according to table 1. Soluble povidone K90 and the antioxidants α-dl-tocopherol, ascorbyl palmitate and sodium metasulfite were dissolved in ethanol, and the mixture was heated to 50 °C. Then, rotigotine was added until dissolved. The silicone adhesives Bio-PSA 7-4302 and Bio-PSA7-4202 were added and mixed uniformly. The mixed solution was coated onto a release film and dried at 40 °C for 4 minutes and at 85 °C for 4 minutes to remove the solvent. In this comparative example, the formulation patch was packaged in an aluminum foil bag containing neither nitrogen gas nor a hygroscopic agent. As shown in table 1, after storage at 40 °C for 6 months, the contents of EP impurity B, EP impurity C and EP impurity K in comparative example A1 were high.

### Comparative Example A2

Each component was weighed according to table 1. The transdermal patch of comparative example A2 was prepared using the same method as comparative example A1. In comparative example A2, the formulation patch was packaged in an aluminum foil bag containing neither nitrogen gas nor a hygroscopic agent. As shown in table 1, after storage at 40 °C for 6 months, the contents of EP impurity B, EP impurity C and EP impurity K in comparative example A2 were relatively high.

### Comparative Example A3

Each component was weighed according to the following weight ratio. 3.33% PVP K90, 0.0200% ascorbyl palmitate, 0.0009% sodium metasulfite, 0.0200% DL-α-tocopherol, 7.5% rotigotine, 13.3694% Bio-PSA 7-4302 and 75.7597% Bio-PSA 7-4202. Soluble povidone K90 and the antioxidants α-dl-tocopherol, ascorbyl palmitate and sodium metasulfite were dissolved in ethanol, and the mixture was heated to 50 °C. Then, rotigotine was added until dissolved. The silicone adhesives Bio-PSA 7-4302 and Bio-PSA7-4202 at room temperature were added and mixed uniformly. The mixed solution was coated onto a release film and dried at 40 °C for 4 minutes and at 85 °C for 4 minutes to remove the solvent. In this comparative example, the formulation patch was packaged in an aluminum foil bag containing neither nitrogen gas nor a hygroscopic agent. After storage at 40 °C for 6 months, the contents of EP impurity B, EP impurity C and EP impurity K in comparative example A3 were relatively high, which was similar to comparative examples A1-A2.

In comparative examples A1-A3, crystallization of rotigotine was observed after storage at room temperature, 40 °C or 60 °C for a period of time.

### Comparative Example A4

Each component was weighed according to table 1. The antioxidants α-dl-tocopherol, ascorbyl palmitate and sodium metasulfite were dissolved in ethanol, and insoluble crospovidone was added and further mixed for 24 hours. The mixed solution was heated to 50 °C, and then rotigotine was added until dissolved. The silicone adhesives Bio-PSA 7-4302 and Bio-PSA7-4202 at room temperature were added and mixed uniformly. The mixed solution was coated onto a release film and dried at 40 °C for 4 minutes and at 85 °C for 4 minutes to remove the solvent. In this comparative example, the formulation patch was packaged in an aluminum foil bag containing neither nitrogen gas nor a hygroscopic agent.

As shown in table 1, due to the high content of crospovidone contained in the formulation, the contents of EP impurity B, EP impurity C, EP impurity E and EP impurity K in comparative example A4 were higher than those in comparative examples A1, A2 and A3. The stability data of comparative examples A1-A4 show that, compared with PVP K90, the use of crospovidone accelerates the degradation of rotigotine in the patch formulation, thereby reducing the stability of the patch formulation.

### Examples A1-A2

Each component was weighed according to table 1. The formulation patches of examples A1-2 were prepared by a method similar to that of comparative example A3. The formulation patches of examples A1-A2 were filled with nitrogen to reduce oxygen to 0.2% and packaged in aluminum foil bags. Compared with comparative examples A1-A3, after being placed at 40 °C for 6 months, the contents of EP impurity B, EP impurity C and EP impurity K in the formulation patch of examples A1-A2 were significantly lower. The results show that on the basis of the use of antioxidant components in a formulation patch, filling a packaging with nitrogen can further inhibit the degradation of rotigotine.

TABLE 1
Stability data of comparative examples A1-4 and examples A1-2

| | | Sample | | | | | |
|---|---|---|---|---|---|---|---|
| | | placement | | EP-B | EP-E | EP-C | EP-K |
| | Formulation information | condition | Time | impurity | impurity | impurity | impurity |
| Comparative | 4.0% PVP K90, 0.02% | Without | 6 months | 0.35 | 0.00 | 0.54 | 0.18 |
| example A1 | ascorbyl palmitate, | N2 | | | | | |
| | 0.0009% sodium | With | | | | | |
| | metasulfite, 0.02% DL-α- | packaging | | | | | |
| | tocopherol, 9.000% | 40 °C | | | | | |
| | rotigotine, 52.1755% Bio- | | | | | | |
| | PSA 7-4302 and 34.7836% | | | | | | |
| | Bio-PSA 7-4202 | | | | | | |
| Comparative | 5.0% PVP K90, 0.02% | Without | 6 months | 0.46 | 0.00 | 0.67 | 0.15 |
| example A2 | ascorbyl palmitate, | N2 | | | | | |
| | 0.0009% sodium | With | | | | | |
| | metasulfite, 0.02% DL-α- | packaging | | | | | |
| | tocopherol, 9.000% | 40 °C | | | | | |
| | rotigotine, 51.5755% Bio- | | | | | | |
| | PSA 7-4302 and 34.3836% | | | | | | |
| | Bio-PSA 7-4202 | | | | | | |
| Comparative | 16.65% crospovidone, | Without | 6 months | 1.26 | 0.09 | 1.53 | 0.68 |
| example A4 | 0.0200% ascorbyl | N2 | | | | | |
| | palmitate, 0.0009% | With | | | | | |
| | sodium metasulfite, | packaging | | | | | |
| | 0.0200% vitamin E, 7.5% | 40 °C | | | | | |
| | rotigotine and 75.8091% | | | | | | |
| | Bio-PSA 7-4302 | | | | | | |
| Example A1 | 3.3332% PVP K90, | With N2 | 5 days | 0.00 | 0.00 | 0.04 | 0.01 |
| | 0.0167% ascorbyl | With | 10 days | 0.00 | 0.00 | 0.05 | 0.01 |
| | palmitate, 0.0015% | packaging | | | | | |
| | sodium metasulfite, | 60 °C | | | | | |
| | 0.0417% DL-α-tocopherol, | With N2 | 3 months | 0.00 | 0.00 | 0.04 | 0.02 |
| | 7.5000% rotigotine, | With | 6 months | 0.04 | 0.02 | 0.06 | 0.02 |
| | 53.4641% Bio-PSA 7-4302 | packaging | | | | | |
| | and 35.6428% Bio-PSA 7- | 40 °C | | | | | |
| | 4202 | With N2 | 3 months | 0.00 | 0.00 | 0.02 | 0.01 |
| | | With | 6 months | 0.02 | 0.04 | 0.05 | 0.01 |
| | | packaging | | | | | |
| | | RT | | | | | |
| Example A2 | 4.0000% PVP K90, | With N2 | 5 days | 0.00 | 0.00 | 0.04 | 0.01 |
| | 0.0167% ascorbyl | With | | | | | |
| | palmitate, 0.0015% | packaging | 10 days | 0.04 | 0.00 | 0.05 | 0.01 |
| | sodium metasulfite, | 60 °C | | | | | |
| | 0.0417% DL-α-tocopherol, | With N2 | 3 months | 0.00 | 0.00 | 0.04 | 0.01 |
| | 9.0000% rotigotine, | With | 6 months | 0.02 | 0.02 | 0.07 | 0.02 |
| | 47.8171% Bio-PSA 7-4302 | packaging | | | | | |
| | and 39.1230% Bio-PSA 7- | 40 °C | | | | | |
| | 4202 | With N2 | 3 months | 0.00 | 0.00 | 0.04 | 0.01 |
| | | With | 6 months | 0.04 | 0.05 | 0.05 | 0.01 |
| | | packaging | | | | | |
| | | RT | | | | | |

### Comparative Example A5 and Examples A3-A6

Each component was weighed according to table 1. Soluble povidone K90 and the antioxidants α-dl-tocopherol, ascorbyl palmitate and sodium metasulfite were dissolved in ethanol, and crospovidone was added and further mixed for 24 hours. The mixed solution was heated to 50 °C, and then rotigotine was added until dissolved. The silicone adhesives Bio-PSA 7-4302 and Bio-PSA7-4202 at room temperature were added and mixed uniformly. The suspension was coated onto a release film and dried at 40 °C for 4 minutes and at 85 °C for 4 minutes to remove the solvent. The formulation patches were packaged using different packaging methods and placed at 60 °C for 5 days. The related substances of comparative example A5 and examples A3-A6 were examined. The experimental conclusions are summarized in table 2.

The formulation patch of comparative example A5 was packaged using only an aluminum foil bag.

The formulation patches of examples A3 were filled with nitrogen to reduce oxygen to 0.2% and packaged in aluminum foil bags. Compared with comparative example A5, the contents of EP impurity B, EP impurity C and EP impurity K in example A3 were reduced.

With regard to the formulation patch of example A4, an iron-based oxygen scavenger not in contact with the patch was placed in a packaging bag, nitrogen was filled to reduce oxygen to 0.2%, and the patch and iron-based oxygen scavenger were packaged in an aluminum foil bag. There is no difference in impurity contents between example A4 and example A3. On the basis of filling a packaging with nitrogen, further addition of an iron-based oxygen scavenger cannot further inhibit the degradation of rotigotine in the formulation.

With regard to the formulation patch of example A5, a hygroscopic film bag containing hygroscopic calcium oxide was placed in a packaging bag, nitrogen was filled to reduce oxygen to 0.2%, and the patch and hygroscopic film bag were packaged in an aluminum foil bag. On the basis of filling a packaging with nitrogen, further addition of a hygroscopic film bag can inhibit the generation of some impurities.

With regard to the formulation patch of example A6, a hygroscopic film bag containing hygroscopic calcium oxide and an iron-based oxygen scavenger were placed in a packaging bag, nitrogen was filled to reduce oxygen to 0.2%, and the patch, hygroscopic film bag and iron-based oxygen scavenger were packaged in an aluminum foil bag. On the basis of filling a packaging with nitrogen, further addition of a hygroscopic film bag and an iron-based oxygen scavenger cannot further improve the degradation of rotigotine in the formulation.

The data in table 2 show that there is little difference in degradation impurity contents among examples A3, A4, A5 and A6. In the transdermal patch formulation using both PVP K90 and cross-linked PVP as crystallization inhibitors, if the amount of the cross-linked PVP is not large, the use of an aluminum foil packaging filled with N2 can inhibit the degradation of rotigotine. On the basis of filling with N2, further addition of a hygroscopic agent, an iron-based oxygen scavenger, or a combination of a hygroscopic agent and an iron-based oxygen scavenger cannot further inhibit the degradation of rotigotine.

TABLE 2
Stability data of comparative example A5 and examples A3-A6

| | | Sample | | EP- | EP- | EP- | EP- |
|---|---|---|---|---|---|---|---|
| | Formulation | placement | | impurity | impurity | impurity | impurity |
| | information | condition | Time | B | E | C | K |
| Comparative | 2.500% PVP K90, | Without | 5 days | 0.09 | 0.00 | 0.22 | 0.15 |
| example A5 | 6.700% crospovidone, | N2 | | | | | |
| | 0.0167% ascorbyl | With | | | | | |
| | palmitate, 0.0015% | packaging | | | | | |
| | sodium metasulfite, | 60 °C | | | | | |
| Example A3 | 0.0583% DL-α- | With N2 | 5 days | 0.07 | 0.05 | 0.08 | 0.10 |
| | tocopherol, 7.5000% | With | | | | | |
| | rotigotine, | packaging | | | | | |
| | 49.9341% Bio-PSA 7- | 60 °C | | | | | |
| Example A4 | 4302 and 33.2894% | With N2 | 5 days | 0.06 | 0.05 | 0.06 | 0.09 |
| | Bio-PSA 7-4202 | With | | | | | |
| | | packaging | | | | | |
| | | Iron-based | | | | | |
| | | oxygen | | | | | |
| | | scavenger | | | | | |
| | | 60 °C | | | | | |
| Example A5 | | With N2 | 5 days | 0.06 | 0.13 | 0.05 | 0.08 |
| | | With | | | | | |
| | | packaging | | | | | |
| | | CaO | | | | | |
| | | 60 °C | | | | | |
| Example A6 | | With N2 | 5 days | 0.06 | 0.09 | 0.08 | 0.10 |
| | | With | | | | | |
| | | packaging | | | | | |
| | | Iron-based | | | | | |
| | | oxygen | | | | | |
| | | scavenger | | | | | |
| | | CaO | | | | | |
| | | 60 °C | | | | | |

### Comparative Examples A6-A10 and Examples A7-A9

Each component of a composition was weighed according to table 3, and the formulation was prepared. The antioxidants ascorbyl palmitate, sodium metasulfite and α-dl-tocopherol were dissolved in isopropanol at 50 °C. Insoluble crospovidone was added and mixed at room temperature for 24 hours. The suspension was heated to 50 °C, and rotigotine was added until completely dissolved. A solution of light mineral oil and polyisobutylene adhesives Oppanol B12 and Oppanol N100 in n-heptane heated to 50 °C was added and mixed. The suspension was kept at 50 °C, coated onto a release film, and dried at 40 °C for 4 min and at 85 °C for 4 min to remove the solvent.

In comparative examples A6-A10 and examples A7-A9, only crospovidone was used as a crystallization inhibitor for rotigotine.

In the aluminum foil bags of comparative examples A6-A10, neither nitrogen nor a hygroscopic agent was used. As shown in table 3, after storage at 40 °C for 3 months, the contents of EP impurity B, EP impurity C, EP impurity E and EP impurity K in comparative examples A6-10 increased significantly.

The patch formulations of examples A7-A9 were filled with nitrogen to reduce oxygen to 0.2% and packaged in aluminum foil bags. As shown in table 3, after storage at 60 °C, 40 °C and room temperature for a period of time, the contents of EP impurity B, EP impurity C, EP impurity E and EP impurity K in examples A7-A9 were lower than those in comparative examples A6-A10. The results show that filling a packaging with nitrogen can inhibit the degradation of rotigotine in a formulation in which only crospovidone was used.

TABLE 3
Stability data of comparative examples A6-A9 and examples A7-A9

| | | Sample | | EP- | EP- | EP- | EP- |
|---|---|---|---|---|---|---|---|
| | | placement | | impurity | impurity | impurity | impurity |
| | Formulation information | condition | Time | B | E | C | K |
| Comparative | 20.0000% crospovidone, | With | 3 | 0.89 | 0.05 | 1.24 | 0.55 |
| example A6 | 0.0200% ascorbyl | packaging | months | | | | |
| | palmitate, 0.0009% | 40 °C | | | | | |
| | sodium metasulfite, | Without | | | | | |
| | 0.0200% DL-α-tocopherol, | N2 | | | | | |
| | 28.3836% light mineral oil, | | | | | | |
| | 25.5453% Oppanol B12, | | | | | | |
| | 17.0302% Oppanol N100 | | | | | | |
| | and 9.0000% rotigotine | | | | | | |
| Comparative | 27.0000% crospovidone, | With | 3 | 0.70 | 0.00 | 1.18 | 1.94 |
| example A7 | 0.0200% ascorbyl | packaging | months | | | | |
| | palmitate, 0.0009% | 40 °C | | | | | |
| | sodium metasulfite, | Without | | | | | |
| | 0.0200% DL-α-tocopherol, | N2 | | | | | |
| | 25.5836% light mineral oil, | | | | | | |
| | 23.0253% Oppanol B12, | | | | | | |
| | 15.3502% Oppanol N100 | | | | | | |
| | and 9.0000% rotigotine | | | | | | |
| Comparative | 16.6500% crospovidone, | With | 3 | 0.70 | 0.07 | 0.70 | 0.57 |
| example A8 | 0.0167% ascorbyl | packaging | months | | | | |
| | palmitate, 0.0015% | Without | | | | | |
| | sodium metasulfite, | N2 | | | | | |
| | 0.0417% DL-α-tocopherol, | 40 °C | | | | | |
| | 26.5265% light mineral oil, | | | | | | |
| | 29.5581% Oppanol B12 | | | | | | |
| | 19.7054% Oppanol N100 | | | | | | |
| | and 7.5000% rotigotine | | | | | | |
| Comparative | 10.0000% crospovidone, | With | 6 | 0.74 | 0.04 | 0.89 | 0.69 |
| example A9 | 0.0167% ascorbyl | packaging | months | | | | |
| | palmitate, 0.0015% | Without | | | | | |
| | sodium metasulfite, | N2 | | | | | |
| | 0.0417% DL-α-tocopherol, | 40 °C | | | | | |
| | 7.5000% rotigotine, | | | | | | |
| | 28.8540% light mineral oil, | | | | | | |
| | 32.1516% Oppanol B12, | | | | | | |
| | 21.4344% Oppanol N100 | | | | | | |
| Example A7 | 16.6500% crospovidone, | With N2 | 5 days | 0.07 | 0.65 | 0.23 | 0.23 |
| | 0.0167% ascorbyl | With | | | | | |
| | palmitate, 0.0015% | packaging | | | | | |
| | sodium metasulfite, | 60 °C | | | | | |
| | 0.0583% DL-α-tocopherol, | With N2 | 1 month | 0.04 | 0.29 | 0.55 | 0.20 |
| | 7.5000% rotigotine, | With | | | | | |
| | 26.5207% light mineral oil, | packaging | | | | | |
| | | 40 °C | | | | | |
| | 29.5517% Oppanol B12 | With N2 | 6 | 0.17 | 0.15 | 0.30 | 0.28 |
| | and 19.7011% Oppanol | With | months | | | | |
| | N100 | packaging | | | | | |
| | | RT | | | | | |
| Example A8 | 20.00% crospovidone, | With N2 | 5 days | 0.07 | 0.55 | 0.27 | 0.26 |
| | 0.0167% ascorbyl | With | | | | | |
| | palmitate, | packaging | | | | | |
| | 0.0015% sodium | 60 °C | | | | | |
| | metasulfite, 0.0583% DL- | With N2 | 1 month | 0.17 | 0.09 | 0.80 | 0.27 |
| | a-tocopherol, 7.5000% | With | | | | | |
| | rotigotine, | packaging | | | | | |
| | 25.3482% light mineral oil, | 40 °C | | | | | |
| | 28.2452% Oppanol B12 | With N2 | 6 | 0.50 | 0.11 | 0.77 | 0.45 |
| | and 18.8301% Oppanol | With | months | | | | |
| | N100 | packaging | | | | | |
| | | RT | | | | | |
| Example A9 | 13% crospovidone, | With N2 | 5 days | 0.02 | 0.22 | 0.17 | 0.22 |
| | 0.0167% ascorbyl | With | | | | | |
| | palmitate, | packaging | | | | | |
| | 0.0015% sodium | 60 °C | | | | | |
| | metasulfite, 0.0583% DL- | With N2 | 1 month | 0.02 | 0.11 | 0.17 | 0.20 |
| | a-tocopherol, 7.5000% | With | | | | | |
| | rotigotine, | packaging | | | | | |
| | 27.7982% light mineral oil, | 40 °C | | | | | |
| | 30.9752% Oppanol B12 | With N2 | 6 | 0.19 | 0.04 | 0.39 | 0.30 |
| | and 20.6501% Oppanol | With | months | | | | |
| | N100 | packaging | | | | | |
| | | RT | | | | | |

### Examples A10-A12

Each component of a composition was weighed according to table 4, and the formulation was prepared. The antioxidants ascorbyl palmitate, sodium metasulfite, DL-α-tocopherol and BHT were dissolved in isopropanol at 50 °C. Insoluble crospovidone was added and mixed at room temperature for 24 hours. The suspension was heated to 50 °C, and rotigotine was added until completely dissolved. A solution of light mineral oil and polyisobutylene adhesives Oppanol B12 and Oppanol N100 in n-heptane heated to 50 °C was added and mixed. The suspension was kept at 50 °C, coated onto a release film, and dried at 40 °C for 4 min and at 85 °C for 4 min to remove the solvent.

The formulation patch of example A10 was filled with nitrogen to reduce oxygen to 0.2% and packaged in an aluminum foil bag. After storage at 60 °C, 40 °C and room temperature for a period of time, the contents of EP impurity B, EP impurity C and EP impurity K were maintained at a relatively low level.

In example A11, a hygroscopic film bag containing silica gel was placed in a packaging bag, nitrogen was filled to reduce oxygen to 0.2%, and the patch and the silica gel-containing hygroscopic film bag were packaged in an aluminum foil bag. On the basis of filling a packaging with nitrogen, further addition of a silica gel-containing hygroscopic film bag can further inhibit the generation of impurities.

In example A12, a hygroscopic film bag containing hygroscopic calcium chloride was placed in a packaging bag, nitrogen was filled to reduce oxygen to 0.2%, and the patch and hygroscopic film bag were packaged in an aluminum foil bag. On the basis of filling a packaging with nitrogen, the addition of a hygroscopic film bag containing hygroscopic calcium chloride can further inhibit the generation of impurities.

Compared with example A10, a hygroscopic agent was used in examples A11-A12, which further inhibited the generation of degradation impurities, especially oxidative degradation impurities and the secondary degradation impurities thereof.

TABLE 4
Stability data of examples A10-12

| | | Sample | | EP- | EP- | EP- | EP- |
|---|---|---|---|---|---|---|---|
| | | placement | | impurity | impurity | impurity | impurity |
| | | condition | Time | B | E | C | K |
| Example | 16.6500% crospovidone, | With N2 | 5 days | 0.00 | 0.33 | 0.13 | 0.25 |
| A10 | 0.0167% ascorbyl palmitate, | With | | | | | |
| | 0.0015% sodium | packaging | | | | | |
| | metasulfite, 0.0583% DL-α- | 60 °C | | | | | |
| | tocopherol, 0.0500% BHT, | With N2 | 1 month | 0.09 | 0.33 | 0.08 | 0.25 |
| | 7.5000% rotigotine, | With | | | | | |
| | 26.5032% light mineral oil, | packaging | | | | | |
| | 29.5322% Oppanol B12, | 40 °C | | | | | |
| | 19.881% Oppanol N100 | With N2 | 3 months | 0.07 | 0.24 | 0.07 | 0.20 |
| | | With | | | | | |
| | | packaging | | | | | |
| | | RT | | | | | |
| Example | | With N2 | 5 days | 0.00 | 0.25 | 0.08 | 0.17 |
| A11 | | With | | | | | |
| | | packaging | | | | | |
| | | 2 g of | | | | | |
| | | silica gel | | | | | |
| | | 60 °C | | | | | |
| | | With N2 | 1 month | 0.09 | 0.24 | 0.10 | 0.15 |
| | | With | | | | | |
| | | packaging | | | | | |
| | | 2 g of | | | | | |
| | | silica gel | | | | | |
| | | 40 °C | | | | | |
| | | With N2 | 3 months | 0.07 | 0.16 | 0.08 | 0.11 |
| | | With | | | | | |
| | | packaging | | | | | |
| | | 2 g of | | | | | |
| | | silica gel | | | | | |
| | | RT | | | | | |
| Example | | With N2 | 5 days | 0.00 | 0.22 | 0.08 | 0.15 |
| A12 | | With | | | | | |
| | | packaging | | | | | |
| | | 2 g of CaCl₂ | | | | | |
| | | 60 °C | | | | | |
| | | With N2 | 1 month | 0.07 | 0.24 | 0.08 | 0.14 |
| | | With | | | | | |
| | | packaging | | | | | |
| | | 2 g of CaCl₂ | | | | | |
| | | 40 °C | | | | | |
| | | With N2 | 3 months | 0.07 | 0.16 | 0.07 | 0.12 |
| | | With | | | | | |
| | | packaging | | | | | |
| | | 2 g of CaCl₂ | | | | | |
| | | RT | | | | | |

### Comparative Example A11 and Examples A13-A16

Each component was weighed according to table 5. The antioxidants ascorbyl palmitate, sodium metasulfite and DL-α-tocopherol were dissolved in isopropanol at 50 °C. Insoluble crospovidone was added and mixed at room temperature for 24 hours. The suspension was heated to 50 °C, and rotigotine was added until completely dissolved. A solution of light mineral oil and polyisobutylene adhesives Oppanol B12 and Oppanol N100 in n-heptane heated to 50 °C was added and mixed. The suspension was kept at 50 °C, coated onto a release film, and dried at 40 °C for 4 min and at 85 °C for 4 min to remove the solvent.

The formulation patches were packaged using different packaging methods and placed at 60 °C for 5 days. The related substances of comparative example A11 and examples A13-A16 were examined. The experimental conclusions are summarized in table 5.

The formulation patch of comparative example A11 was packaged using only an aluminum foil bag.

The formulation patch of example A13 was filled with nitrogen to reduce oxygen to 0.2% and packaged in an aluminum foil bag. Compared with comparative example A11, the contents of EP impurity B, EP impurity C and EP impurity K in example A13 were reduced.

With regard to the formulation patch of example A14, an iron-based oxygen scavenger was placed in a packaging bag, nitrogen was filled to reduce oxygen to 0.2%, and the patch and iron-based oxygen scavenger were packaged in an aluminum foil bag. There is no difference in impurity contents between example A14 and example A13. On the basis of filling a packaging with nitrogen, further addition of an iron-based oxygen scavenger cannot further inhibit the degradation of rotigotine in the formulation.

With regard to the formulation patch of example A15, a hygroscopic film bag containing hygroscopic calcium oxide was placed in a packaging bag, nitrogen was filled to reduce oxygen to 0.2%, and the patch and hygroscopic film bag were packaged in an aluminum foil bag. On the basis of filling a packaging with nitrogen, further addition of a hygroscopic film bag can inhibit the generation of four impurities.

With regard to the formulation patch of example A16, a hygroscopic film bag containing hygroscopic calcium oxide and an iron-based oxygen scavenger were placed in a packaging bag, nitrogen was filled to reduce oxygen to 0.2%, and the patch, hygroscopic film bag and iron-based oxygen scavenger were packaged in an aluminum foil bag. The levels of related impurities of example A16 and example A15 were similar. It shows that on the basis of filling a packaging with nitrogen, further addition of a hygroscopic film bag and an iron-based oxygen scavenger cannot further improve the degradation of rotigotine in the formulation.

The data in table 5 show that in a transdermal patch formulation using only cross-linked PVP as a rotigotine crystallization inhibitor, the use of an aluminum foil packaging filled with N2 can inhibit the degradation of rotigotine. If the amount of cross-linked PVP is large, on the basis of filling with N2, the addition of a hygroscopic agent can further inhibit the degradation of rotigotine. Further addition of an iron-based oxygen scavenger to a packaging cannot further improve the stability of rotigotine.

TABLE 5
Stability data of comparative example A11 and examples A13-A16

| | | Sample | | EP- | EP- | EP- | EP- |
|---|---|---|---|---|---|---|---|
| | Formulation | placement | | impurity | impurity | impurity | impurity |
| | information | condition | Time | B | E | C | K |
| Comparative | 17.5000% | Without | 5 days | 0.41 | 0.05 | 0.35 | 0.29 |
| example A11 | crospovidone, | N2 | | | | | |
| | 0.0167% ascorbyl | With | | | | | |
| | palmitate, 0.0015% | packaging | | | | | |
| | sodium metasulfite, | 60 °C | | | | | |
| Example A13 | 0.0583% DL-α- | N2 | 5 days | 0.17 | 0.18 | 0.13 | 0.23 |
| | tocopherol, | With | | | | | |
| | 7.5000% rotigotine, | packaging | | | | | |
| | 26.2232% light | 60 °C | | | | | |
| Example A14 | mineral oil, | With N2 | 5 days | 0.19 | 0.18 | 0.13 | 0.23 |
| | 29.2202% Oppanol | With | | | | | |
| | B12 and 19.4801% | packaging | | | | | |
| | Oppanol N100 | Iron-based | | | | | |
| | | oxygen | | | | | |
| | | scavenger | | | | | |
| | | 60°C | | | | | |
| Example A15 | | With N2 | 5 days | 0.13 | 0.15 | 0.10 | 0.14 |
| | | With | | | | | |
| | | packaging | | | | | |
| | | CaO | | | | | |
| | | 60 °C | | | | | |
| Example A16 | | With N2 | 5 days | 0.13 | 0.15 | 0.11 | 0.14 |
| | | With | | | | | |
| | | packaging | | | | | |
| | | Iron-based | | | | | |
| | | oxygen | | | | | |
| | | scavenger | | | | | |
| | | CaO | | | | | |
| | | 60°C | | | | | |

### Comparative Example B1 and Examples B1-B4

K 90, oleic acid, EPO, DL-α-tocopherol, 0.002% ascorbyl palmitate NF, 0.1% BHT and risperidone were mixed with ethanol. Crospovidone was added and mixed for 24 hours. Duro-Tak 387-2516 was added and mixed uniformly. The wet blend was coated onto a release liner, dried, and then laminated to the Scotchpak 1109 backing. The laminated material was die-cut into individual patches.

The patches were packaged using different packaging methods and placed at 60 °C for 10 days. The contents of risperidone N-oxide (RRT 0.64), unknown impurities (RRT 0.88) and total impurities of comparative example B1 and examples B1-B4 were examined. The measurement results are summarized in table 6.

The patch of comparative example B1 was directly packaged in an aluminum foil bag, in which neither nitrogen nor a hygroscopic agent nor an iron-based oxygen scavenger was used. After being placed at 60 °C for 10 days, the contents of N-oxide impurities (RRT 0.64) and unknown impurities (RRT 0.88) were high, and the total impurity content was 1.28%.

The patch of example B1 was filled with nitrogen and packaged in an aluminum foil bag. After being placed at 60 °C for 10 days, the contents of N-oxide impurities and total impurities of the patch of example B1 were significantly reduced compared with comparative example B1.

The patch of example B2 was packaged in an aluminum foil bag along with an iron-based oxygen scavenger and nitrogen. After being placed at 60 °C for 10 days, the content of N-oxide impurities was reduced compared with comparative example B1.

The patch of example B3 was packaged in an aluminum foil bag along with a calcium oxide-based inner hygroscopic film bag and nitrogen. After being placed at 60 °C for 10 days, the content of N-oxide impurities was significantly reduced compared with comparative example B1 and example B2.

The patch of example B4 was packaged in an aluminum foil bag along with an iron-based oxygen scavenger, a calcium oxide-based inner hygroscopic film bag and nitrogen. The content of N-oxide impurities was significantly reduced compared with comparative example B1 and example B2.

Based on the test results of examples B3 and B4, the calcium oxide-based inner hygroscopic film bag and nitrogen filling can significantly reduce risperidone impurities, especially the content of risperidone N-oxides.

TABLE 6
Stability data of comparative example B1 and examples B1-B4

| | Sample | | | Unknown | Total |
|---|---|---|---|---|---|
| | placement | | N-oxide | impurity | im- |
| | condition | Time | RRT0.64 | RRT0.88 | purity |
| Comparative | Without N2, with | 10 days | 1.14% | 0.14% | 1.28% |
| example B1 | packaging, 60 °C. | | | | |
| Example B1 | With N2, with | 10 days | 0.72% | 0.11% | 0.83% |
| | packaging, 60 °C. | | | | |
| Example B2 | With N2, with | 10 days | 0.95% | < 0.05% | 0.95% |
| | packaging, 0.4 g | | | | |
| | of iron-based | | | | |
| | oxygen | | | | |
| | scavenger, 60 °C. | | | | |
| Example B3 | With N2, with | 10 days | 0.08% | < 0.05% | 0.08% |
| | packaging, CaO, | | | | |
| | 60 °C. | | | | |
| Example B4 | With N2, with | 10 days | < 0.05% | < 0.05% | 0.00% |
| | packaging, 0.4 g | | | | |
| | of iron-based | | | | |
| | oxygen | | | | |
| | scavengers, CaO, | | | | |
| | 60 °C. | | | | |

### Stability Data of Comparative Examples C1-C6 and Examples C1-C7

Each component was weighed according to table 7. Oleic acid, DL-α-tocopherol, ascorbyl palmitate NF, BHT, sodium metasulfite NF and Eudragit E100 were dissolved in ethanol, and crospovidone was added and mixed for 24 hours. Olanzapine was added and mixed until dissolved. Duro-Tak 387-2516 or 387-2287 was added and mixed uniformly. The wet blend was coated onto a release liner, dried, and then laminated to the backing. The laminated material was die-cut into individual patches. The die-cut patches were packaged using different packaging methods. The patches in different packaging were placed at 60 °C for 5 days. The related substances of comparative examples C1-C6 and examples C1-C7 were examined. The experimental conclusions are summarized in table 7.

The aluminum foil packaging without N₂ filling was used in comparative examples C1-C6. Compared with comparative examples C1-C2 in which no antioxidant was added to the matrix of the patch, comparative examples C3-C6 in which an antioxidant was added to the matrix showed an inhibition on the generation of related substances. The antioxidant combination of DL-α-tocopherol, ascorbyl palmitate and sodium metasulfite, and the antioxidant combination of DL-α-tocopherol, ascorbyl palmitate, sodium metasulfite and BHT achieved relatively good effects.

With regard to examples C1-C7, on the basis of filling aluminum foil bags with nitrogen, a hygroscopic agent (CaCl₂ or silica gel) not in contact with the patch was added to the packaging, and the hygroscopic agent not in contact with the patch significantly inhibited the generation of related substances, particularly the generation of EP-impurity D (N-oxide). Preparing the patches of examples C5-C7 from dried raw and auxiliary materials can further reduce the generation of EP-impurity D (N-oxide).

TABLE 7
Stability data of comparative examples C1-C6 and examples C1-C7

| | | | | | | | EP- | | |
|---|---|---|---|---|---|---|---|---|---|
| | | | | | | | impurity | Any other | |
| | | | | Unknown | EP- B | Unknown | D | largest | |
| | | Sample | | impurity | impurity | impurity | N-oxide | single | |
| | Formulation | placement | | RRT | RRT | RRT | RRT | unknown | Total |
| | information | condition | Time | 0.15 | 0.19 | 0.73 | 0.98 | impurity | impurity |
| Comparative | 15% crospovidone | Without | 5 days | ND | 1.42 | 0.43 | 1.64 | 0.29 | 3.78 |
| example C1 | 10% olanzapine, | N2 | | | | | | | |
| | CLM, 75% Dura- | With | | | | | | | |
| | tak 387-2516 | packaging | | | | | | | |
| | | 60 °C | | | | | | | |
| Comparative | 10% olanzapine, | Without | 5 days | 1.57 | 5.07 | 1.81 | 2.21 | 0.85 | 11.74 |
| example C2 | 9.3% oleic acid, | N2 | | | | | | | |
| | 15% crospovidone | With | | | | | | | |
| | CLM, 65.7% Dura- | packaging | | | | | | | |
| | Tak387-2516 | 60 °C | | | | | | | |
| Comparative | 10% olanzapine, | Without | 5 days | 0.78 | 4.43 | 1.32 | 1.70 | 0.31 | 9.23 |
| example C3 | 9.5% oleic acid, | N2 | | | | | | | |
| | 15% crospovidone | With | | | | | | | |
| | CLM, 0.20% DL-α- | packaging | | | | | | | |
| | tocopherol, 65.3% | 60 °C | | | | | | | |
| | Dura-Tak-387- | | | | | | | | |
| | 2516 | | | | | | | | |
| Comparative | 10% olanzapine, | Without | 5 days | 0.75 | 3.70 | 1.3 | 0.66 | 0.25 | 7.02 |
| example C4 | 10% oleic acid, | N2 | | | | | | | |
| | 15% Eudragit | With | | | | | | | |
| | E100, 0.20% DL-α- | packaging | | | | | | | |
| | tocopherol, 64.8% | 60 °C | | | | | | | |
| | Dura-Tak 387- | | | | | | | | |
| | 2516 | | | | | | | | |
| Comparative | 10% olanzapine, | Without | 5 days | ND | 0.16 | 0.05 | 0.29 | 0.19 | 0.74 |
| example C5 | 15% Eudragit | N2 | | | | | | | |
| | E100, 0.3% DL-α- | With | | | | | | | |
| | tocopherol, | packaging | | | | | | | |
| | 0.1% ascorbyl | 60 °C | | | | | | | |
| Example C1 | palmitate NF, | With N2 | 5 days | ND | <0.05% | ND | 0.10 | <0.05 | 0.10 |
| | 0.005% sodium | With | | | | | | | |
| | metasulfite NF, | packaging | | | | | | | |
| | 74.60% Dura-Tak | CaCl₂ | | | | | | | |
| | 387-2287 | 60 °C | | | | | | | |
| Comparative | 10% olanzapine, | With | 5 days | 0.87 | 1.95 | 0.96 | 0.71 | 0.11 | 5.21 |
| example C6 | 9.30% oleic acid, | packaging | | | | | | | |
| | 15% Eudragit | 60 °C | | | | | | | |
| Example C2 | E100, 0.3% DL-α- | With N2 | 5 days | 0.22 | 0.10 | ND | 0.14 | <0.05 | 0.46 |
| | tocopherol, 0.1% | With | | | | | | | |
| | ascorbyl palmitate | packaging | | | | | | | |
| | NF, 0.005% | CaCl₂ | | | | | | | |
| | sodium | 60 °C | | | | | | | |
| | metasulfite NF, | | | | | | | | |
| | 65.30% Dura-Tak | | | | | | | | |
| | 387-2287 | | | | | | | | |
| Example C3 | 10% olanzapine, | With N2 | 5 days | ND | <0.05% | ND | 0.17 | <0.05 | 0.17 |
| | Eudragit E100 | With | | | | | | | |
| | 15%, 0.3% DL-α- | packaging | | | | | | | |
| | tocopherol, | Silica gel | | | | | | | |
| | 0.020% ascorbyl | 60°C | | | | | | | |
| | palmitate NF, | | | | | | | | |
| | 0.0005% sodium | | | | | | | | |
| | metasulfite NF, | | | | | | | | |
| | 74.68% Dura-Tak | | | | | | | | |
| | 387-2287 | | | | | | | | |
| Example C4 | 10% olanzapine, | With N2 | 5 days | 0.22 | 0.10 | ND | 0.14 | <0.05 | 0.46 |
| | 9.30% oleic acid, | With | | | | | | | |
| | 15% Eudragit | packaging | | | | | | | |
| | E100, 0.3% DL-α- | CaCl₂ | | | | | | | |
| | tocopherol, 0.1% | 60 °C | | | | | | | |
| | ascorbyl palmitate | | | | | | | | |
| | NF, 0.005% | | | | | | | | |
| | sodium | | | | | | | | |
| | metasulfite NF, | | | | | | | | |
| | 65.30% Dura-Tak | | | | | | | | |
| | 387-2287 | | | | | | | | |
| Example C5 | 10% Olan, 10% | With N2 | 5 days | 0.14 | 0.09 | <0.05% | 0.10 | 0.06 | 0.39 |
| | oleic acid, 25% | With | | | | | | | |
| | Eudragit E100, | packaging | | | | | | | |
| | 0.3% DL-α- | CaCl₂ | | | | | | | |
| | tocopherol, | 60 °C | | | | | | | |
| | 0.002% ascorbyl | | | | | | | | |
| | palmitate NF, | | | | | | | | |
| | 0.0015% sodium | | | | | | | | |
| | metasulfite NF, | | | | | | | | |
| | 0.1% BHT, | | | | | | | | |
| | 54.5965% Dura- | | | | | | | | |
| | Tak387-2287 (raw | | | | | | | | |
| | and auxiliary | | | | | | | | |
| | materials dried in | | | | | | | | |
| | a dryer are used) | | | | | | | | |
| Example C6 | 10% Olan, 10% | With N2 | 5 days | 0.08 | 0.06 | <0.05% | 0.09 | <0.05% | 0.23 |
| | oleic acid, 30% | With | | | | | | | |
| | Eudragit E100, | packaging | | | | | | | |
| | 0.3% DL-α- | CaCl₂ | | | | | | | |
| | tocopherol, | 60°C | | | | | | | |
| | 0.002% ascorbyl | | | | | | | | |
| | palmitate NF, | | | | | | | | |
| | 0.0015% sodium | | | | | | | | |
| | metasulfite NF, | | | | | | | | |
| | 0.1% BHT, | | | | | | | | |
| | 49.5965% Dura- | | | | | | | | |
| | Tak387-2287 (raw | | | | | | | | |
| | and auxiliary | | | | | | | | |
| | materials dried in | | | | | | | | |
| | a dryer are used) | | | | | | | | |
| Example C7 | 10% olanzapine, | With N2 | 5 days | 0.16 | 0.13 | <0.05% | 0.11 | 0.09 | 0.40 |
| | 15% oleic acid, | With | | | | | | | |
| | 25% Eudragit | packaging | | | | | | | |
| | E100, 0.3% DL-α- | CaCl₂ | | | | | | | |
| | tocopherol, | 60 °C | | | | | | | |
| | 0.002% ascorbyl | | | | | | | | |
| | palmitate NF, | | | | | | | | |
| | 0.0015% sodium | | | | | | | | |
| | metasulfite NF, | | | | | | | | |
| | 0.1% BHT, | | | | | | | | |
| | 49.5965% Dura- | | | | | | | | |
| | Tak 387-2287 (raw | | | | | | | | |
| | and auxiliary | | | | | | | | |
| | materials dried in | | | | | | | | |
| | a dryer are used) | | | | | | | | |

1. A transdermal drug delivery kit comprising a transdermal drug delivery patch, wherein the transdermal drug delivery patch comprises:
1) a backing layer;
2) a matrix layer containing a drug or a pharmaceutically acceptable salt thereof dispersed in a non-crystalline or partially crystalline state in the matrix layer, an antioxidant, and a pressure-sensitive adhesive, and optionally containing a stabilizer for inhibiting drug crystallization, wherein the drug can be oxidized and degraded, and is preferably oxidized and degraded to generate nitrogen oxide impurities; and
3) a release layer;
wherein the transdermal drug delivery patch is packaged in a packaging container, and the packaging container is filled with an inert gas.

2. The transdermal drug delivery kit according to claim 1, wherein the inert gas is preferably nitrogen; preferably, nitrogen is filled to reduce oxygen to below about 1.0%; more preferably, nitrogen is filled to reduce oxygen to below about 0.5%, 0.2%, 0.15%, 0.1%, 0.05%, or 0.01%.

3. The transdermal drug delivery kit according to claim 1 or 2, wherein a hygroscopic agent not in contact with the matrix layer of the patch is further added to the packaging container.

4. The transdermal drug delivery kit according to claim 3, wherein the hygroscopic agent is selected from one or more of calcium chloride, calcium oxide, barium oxide, calcium sulfate, molecular sieve, sodium sulfate, magnesium sulfate, silicon dioxide, silica gel, potassium hydroxide, potassium carbonate and phosphorus pentoxide.

5. The transdermal drug delivery kit according to any one of claims 1-4, wherein an oxygen scavenger not in contact with the matrix layer of the patch is further added to the packaging container.

6. The transdermal drug delivery kit according to claim 5, wherein the oxygen scavenger not in contact with the matrix layer of the patch comprises a main component selected from a combination of iron powder and sodium chloride, a combination of ferrous carbonate and metal halide, and a combination of ascorbate and sodium bicarbonate, preferably a combination of iron powder and sodium chloride.

7. The transdermal drug delivery kit according to any one of claims 3-6, wherein the hygroscopic agent and/or oxygen scavenger are separately packaged in a breathable film; preferably, the hygroscopic agent and/or oxygen scavenger packaged in the breathable film are separately adhered to or placed on a heat-sealing layer in the packaging container, or are uniformly contained in the heat-sealing layer.

8. The transdermal drug delivery kit according to any one of claims 1-7, wherein the packaging container has low oxygen permeability and low moisture permeability, and is preferably a metal tray, a plastic tray, or a packaging made of a multi-layer laminated material comprising a plastic or paper layer, an aluminum foil layer and a heat-sealable inner layer.

9. The transdermal drug delivery kit according to any one of claims 1-8, wherein the stabilizer for inhibiting drug crystallization and/or the pressure-sensitive adhesive contains a peroxide; preferably, the peroxide-containing stabilizer for inhibiting drug crystallization is selected from insoluble crospovidone, an acrylic acid polymer or a methacrylic acid polymer, and the peroxide-containing pressure-sensitive adhesive is an acrylic pressure-sensitive adhesive; further preferably, the insoluble crospovidone is selected from one or more of insoluble crospovidone CL-M, crospovidone CL, crospovidone CL-F and crospovidone CL-SF, and the peroxide-containing acrylic acid polymer or methacrylic acid polymer for inhibiting drug crystallization is selected from one or more of Eudragit E100, Eudragit PO, Plastoid B, Eudragit S, Eudragit L and Eudragit L-55.

10. The transdermal drug delivery kit according to claim 9, wherein on the basis of the peroxide-containing stabilizer for inhibiting drug crystallization, the stabilizer for inhibiting drug crystallization further comprises a second stabilizer for inhibiting drug crystallization which is different from a first stabilizer for inhibiting drug crystallization, wherein the second stabilizer for inhibiting drug crystallization is selected from one or more of povidone, copovidone, crospovidone, carbohydrate polymers such as hydroxymethyl cellulose, methyl hydroxymethyl acetate, hydroxymethylcellulose phthalate, β-hydroxypropyl cyclodextrin, ethyl cellulose, methyl cellulose, Katasong, hyaluronic acid, pectin, carboxymethyl cellulose, alginic acid and carrageenan; acrylic acid or methacrylic acid polymers such as Eudragit E100, Eudragit PO, Plastoid B, Eudragit S, Eudragit L and Eudragit L-55; more preferably, the second stabilizer for inhibiting drug crystallization is selected from one or more of soluble povidone, Eudragit E100 or Eudragit PO; the povidone is preferably selected from one or more of povidone K30, povidone K90, povidone K12, povidone K17, povidone K25, plasdone K29/32 and copovidone VA64.

11. The transdermal drug delivery kit according to any one of claims 1-10, wherein the antioxidant is selected from one or more of ascorbic acid, ascorbyl palmitate, butylated hydroxyanisole, butylated hydroxytoluene, hypophosphorous acid, monothioglycerol, potassium metabisulfite, propyl gallate, sodium bisulfite, sodium formaldehyde sulfoxylate, sodium metabisulfite, sodium thiosulfite, sodium thiosulfite and DL-α-tocopherol; preferably, the antioxidant further comprises a chelating agent as a synergistic antioxidant, including one or more of citric acid, tartaric acid, calcium disodium edetate, disodium edetate and EDTA; the antioxidant is preferably a combination of ascorbyl palmitate, sodium metasulfite and DL-α-tocopherol, or a combination of ascorbyl palmitate, sodium metasulfite, DL-α-tocopherol and BHT.

12. The transdermal drug delivery kit according to any one of claims 1-11, wherein the matrix layer comprises the following components, relative to the total weight of the matrix layer:
1) the drug or pharmaceutically acceptable salt thereof at an amount of 3%-30%;
2) the stabilizer for inhibiting drug crystallization at an amount of 0%-40%;
3) the antioxidant at an amount of 0.001%-10%;
4) the pressure-sensitive adhesive at an amount of 30%-95%;
5) a tackifier at an amount of 0%-50%;
6) a skin penetration enhancer at an amount of 0%-30%;
7) a cohesion-promoting additive at an amount of 0%-20%;
the total amount of each component in the matrix layer is 100%.

13. The transdermal drug delivery kit according to any one of claims 1-12, wherein the pressure-sensitive adhesive is selected from one or more of an acrylic adhesive, a silicone adhesive, an acrylic-silicone copolymer adhesive, a polybutylene adhesive, a styrene-isoprene-butylene copolymer and a styrene-butadiene-styrene copolymer.

14. The transdermal drug delivery kit according to any one of claims 12 and 13, wherein the tackifier is selected from silicone oil, light mineral oil, polybutylene, triethyl citrate, terpene and a mixture thereof.

15. The transdermal drug delivery kit according to any one of claims 1-14, wherein the drug that can be degraded to generate nitrogen oxide impurities is selected from olanzapine, rotigotine, donepezil, almotriptan, apixaban, aripiprazole, asenapine, baricitinib, bisoprolol, blonanserin, buprenorphine, cariprazine, dextroamphetamine, dexmedetomidine, eletriptan, escitalopram, fingolimod, frovatriptan, granisetron, lasmiditan, lenalidomide, naratriptan, oxybutynin, ozanimod, piroxicam, pramipexole, risperidone, rizatriptan, ropinirole, scopolamine, sumatriptan, tulobuterol and zolmitriptan, or a pharmaceutically acceptable salt thereof.

16. A method for preparing the transdermal drug delivery kit according to any one of claims 1-14, comprising the following steps:
step 1: dissolving an antioxidant in a solvent, optionally dissolving or suspending a stabilizer for inhibiting drug crystallization in the solvent, and mixing for 0.1 hours to 24 hours; preferably, the solvent includes but is not limited to one or more of toluene, ethanol, isopropanol, dimethylacetamide, dimethyl sulfoxide and n-heptane, and more preferably, the solvent is toluene, ethanol, isopropanol, n-heptane, or a mixed solvent thereof;
step 2: adding a drug or a pharmaceutically acceptable salt thereof, and mixing and dissolving until the drug or pharmaceutically acceptable salt thereof is dispersed in a non-crystalline state or in a crystalline state;
step 3: adding a pressure-sensitive adhesive and mixing uniformly to obtain a drug wet mixture;
step 4: coating the drug wet mixture onto a release film;
step 5: drying to remove the solvent so as to obtain a release film/matrix layer laminated material;
step 6: laminating the matrix layer onto the backing layer, and performing die-cutting on a composite film to obtain individual patches;
step 7: filling a packaging container with an inert gas, and sealing and packaging the inert gas, a hygroscopic agent and/or an oxygen scavenger and the cut individual transdermal patch into the container.

17. The method according to claim 16, wherein the drug or pharmaceutically acceptable salt thereof, the pressure-sensitive adhesive, the skin penetration enhancer and the antioxidant are pre-dried.

18. The method according to claim 16 or 17, wherein the hygroscopic agent and/or oxygen scavenger are separately packaged in a breathable film; preferably, the hygroscopic agent and/or oxygen scavenger packaged in the breathable film are separately adhered to or placed on a heat-sealing layer in the packaging container, or are uniformly contained in the heat-sealing layer.

19. Use of the transdermal drug delivery kit according to any one of claims 1-18 in the preparation of a medicament for treating or preventing a disease.

20. A method for treating or preventing a disease, comprising administering a therapeutically effective amount of the transdermal drug delivery kit according to any one of claims 1-18 to a subject in need thereof.
